(19) United States
(12) Patent Application Publication
(10) Pub. No.: US 2010/0094702 A1
(43) Pub. Date: Apr. 15, 2010

(54) METHOD FOR EVALUATING THE EFFECTIVENESS OF COMMERCIAL COMMUNICATION

(76) Inventor: Richard Bernard Silberstein, Victoria (AU)

Correspondence Address: MASTERMIND IP LAW PC, 421-A SANTA MARINA COURT, ESCONDIDO, CA, 92029, US

(21) Appl. No.: 12/520,863

(22) PCT Filed: Dec. 22, 2006
(86) PCT No.: PCT/AU2006/002006

§ 371 Date: Jun. 22, 2009

(51) Int. Cl. G06Q 30/00; G06Q 10/00; A61B 5/0476

(52) U.S. Cl. 705/14.43; 705/14.44; 600/544

(57) ABSTRACT

A method of quantitatively assessing the effectiveness of an audiovisual, visual or audio advertisement including the steps of: presenting the advertisement to a plurality of subjects, the advertisement having a sequence of audiovisual, visual and/or audio features which occur as a function of time; obtaining, during presentation of the advertisement, EEG signals from the subjects from predetermined scalp sites thereof; calculating SSVEP amplitudes and/or phase differences from EEG signals obtained from the predetermined scalp sites in order to obtain output signals which represent predetermined psychological states of each subject to the features as a function of time; combining the output signals from the subjects to obtain pooled output signals; and displaying the pooled output signals to thereby enable quantitative assessment of the subjects' responses to the features of the advertisement in order to assess the effectiveness of the features of the advertisement.

### BACKGROUND OF THE INVENTION

The effectiveness of any piece of commercial communication or advertising depends on whether the appropriate psychological states intended by the advertiser are elicited in the viewer while they perceive the commercial communication, henceforth referred to as advertisement. For example, advertisements require a level of memory encoding at the time that the advertisers' brand appears. Frequently, the advertisement may be designed to elicit particular emotional responses in the target audience. Determining whether advertisements are effective before they are launched is most frequently carried out using a variety of psychological methodologies such as in-depth interviews and focus groups. These are widely considered inadequate in that they rely on the verbal responses of the subjects. Such verbal responses are now considered a poor indicator of emotional states and the market research industry is considering techniques that rely on brain activity measurements.

Over the last 20 years, neuroscience researchers have learnt more about the specialized role of different brain regions associated with specific psychological states or processes. By measuring the activity in different brain regions while participants perceive advertisements, it is possible to infer the psychological state elicited by the advertisement.

This invention relates to a method and system to identify the psychological state experienced by the subject while perceiving an advertisement. Brain activity is measured using a technique termed Steady State Probe Topography (SSPT). A method is disclosed that uses SSPT to determine the cognitive and emotional states that are elicited by the advertisements and in particular, particular points in time during the advertisement. Effectiveness is then defined in terms of whether the psychological states elicited by the advertisements are those intended by the creator of the advertisement. In particular, the most likely measure of effectiveness is the level of memory encoding during the branding component of the advertisement, i.e. when the brand is displayed during the advertisement.

U.S. Pat. Nos. 4,955,938, 5,331,969 and 6,792,304 (the contents of which are hereby incorporated herein by reference) disclose techniques for obtaining a steady state visually evoked potential (SSVEP) from a subject. These patents disclose the use of Fourier analysis in order to rapidly obtain the SSVEP's and changes thereto.

Reference is made to articles entitled Brain-Imaging Detection of Visual Scene Encoding in Long-Term Memory for TV Commercials, John R. Rossiter and Richard B. Silberstein, March/April 2001 Journal of Advertising Research, Pages 13-21 and Frontal Steady-State Potential Changes Predict Long-Term Recognition Memory Performance by Richard B. Silberstein, Philip G. Harris, Geoffrey A. Nield and Andrew Pipingas, International Journal of Psycho Physiology 39 (2000) 79-85, the content of these articles are incorporated herein by cross-reference. The first article describes an experiment using steady state probe topography (SSPT), which is a technique that involves presenting a visual or auditory or combined task such as an advertisment whilst subjects view a peripheral flickering light. In the experiment, subjects were tested for long term memory recall of static frames from TV commercials presented to the subjects one week after viewing the commercials. The article suggests that recall of static frames from the commercial by subjects provides a measure of evaluation of the success of the TV commercial.

It has now been appreciated that the effecitveness of commercial communications or advertising can be evaluated by analysis of responses in different regions of the brain so as to identify predetermined psychological states elicited by the participants as they perceive the commercial communication or advertisment.

In this specification the expression “predetermined psycholgical states” includes the following:

visual attention to detail;

visual attention to global features;

multi-modal attention to detail or desirability;

multi-modal attention to global features or desirability;

emotional intensity;

attraction-repulsion;

engagement; and

behavioural intent.

The expression does not include long term memory encoding per se because long term memory encoding is not considered to be a psychological state for the purposes of the methods of assessment described herein.

### SUMMARY OF THE INVENTION

According to the present invention there is provided a method of quantitatively assessing the effectiveness of an audiovisual, visual or audio advertisement including the steps of:

presenting the advertisement to a plurality of subjects, the advertisement having a sequence of audiovisual, visual and/or audio features which occur as a function of time;

obtaining, during presentation of the advertisement, EEG signals from the subjects from predetermined scalp sites thereof;

calculating SSVEP amplitudes and/or phase differences from EEG signals obtained from said predetermined scalp sites in order to obtain output signals which represent predetermined psychological states of each subject to said features as a function of time;

combining the output signals from said subjects to obtain pooled output signals; and

displaying the pooled output signals to thereby enable quantitative assessment of the subjects' responses to said features of the advertisement in order to assess the effectiveness of the features of the advertisement.

The invention also provides a system for quantitatively assessing the effectiveness of an audiovisual, visual or audio advertisement including:

display means for presenting the advertisement to a plurality of subjects, the advertisement having a sequence of audiovisual, visual or audio features which occur as a function of time;

means for obtaining, during presentation of the advertisement, EEG signals from said at least one subject from predetermined scalp sites of said subjects; and

means for calculating SSVEP amplitudes and/or phase differences from signals obtained from the predetermined sites in order to obtain output signals which represent said predetermined psychological states of said at least one subject to said features as a function of time, to thereby enable quantitative assessment of said subjects' responses to said features of the advertisement in order to assess the effectiveness of the features of the advertisement.

The invention also provides a method of measuring steady-state visually evoked potential (SSVEP) of a subject including the step of applying time varying flicker signals only to the peripheral vision regions of the retina of a subject and not applying said time varying flicker signals to the centre of vision (fovea) of the subject.

### DETAILED DESCRIPTION OF THE INVENTION

The invention will now be further described with reference to the accompanying drawings, in which:

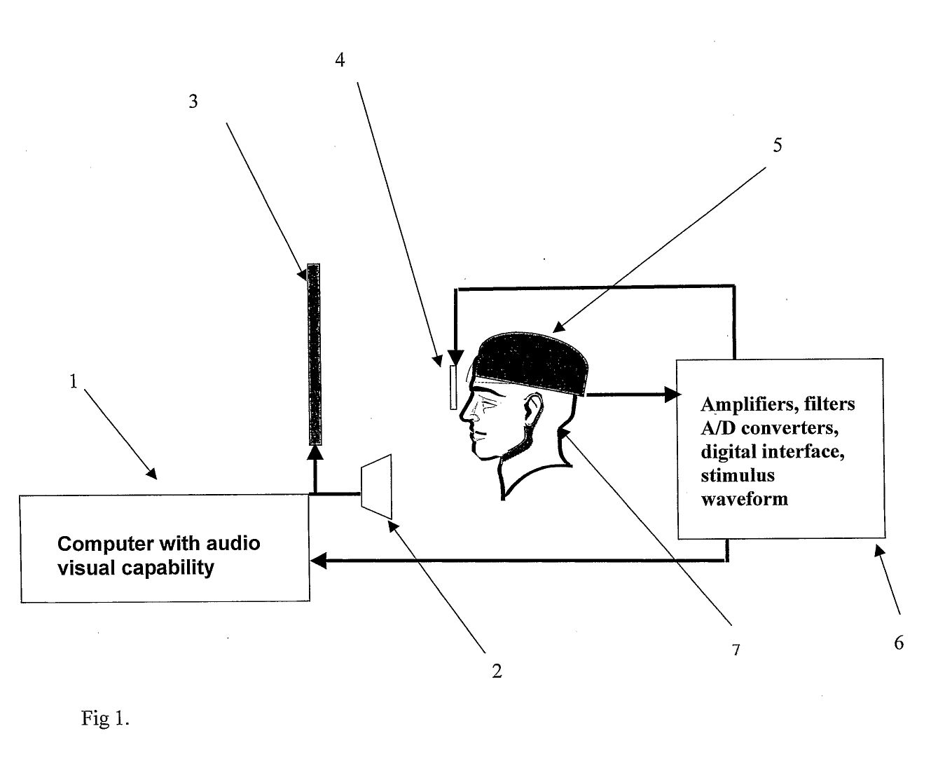

FIG. 1 is a schematic diagram of a system of the invention;

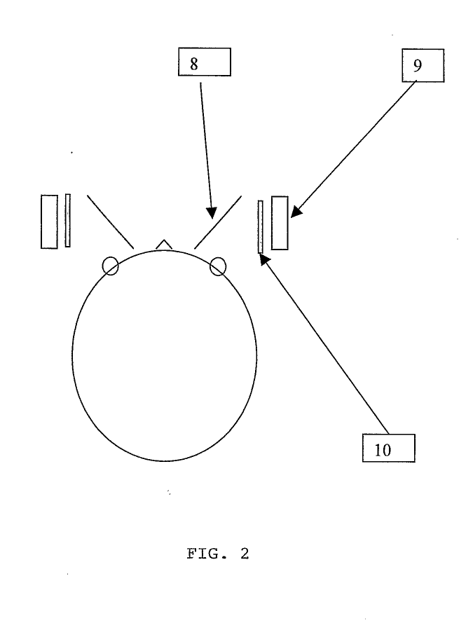

FIG. 2 is a schematic view showing in more detail the manner in which visual flicker stimuli are presented to a subject;

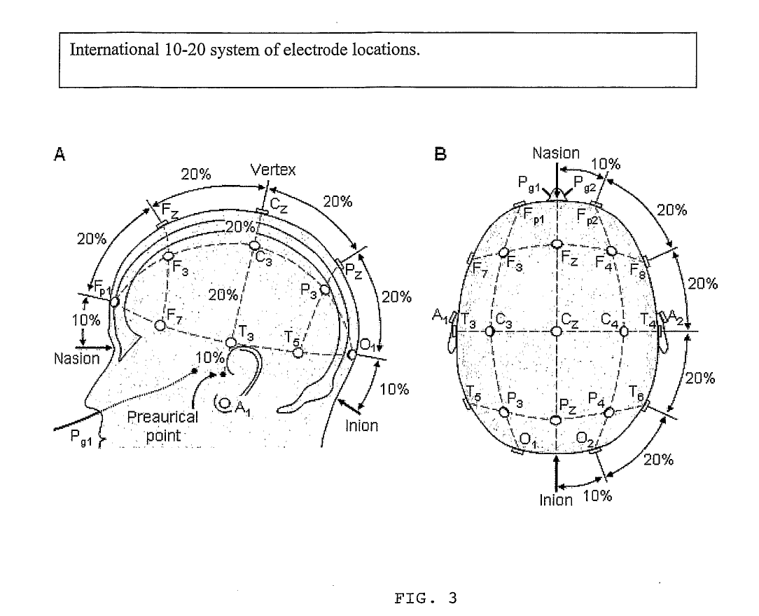

FIG. 3 is a schematic view illustrating the International 10-20 System of electrode locations;

FIG. 4 is a diagrammatic representation showing opacity as a function of radius of a screen which is used in the system of the invention;

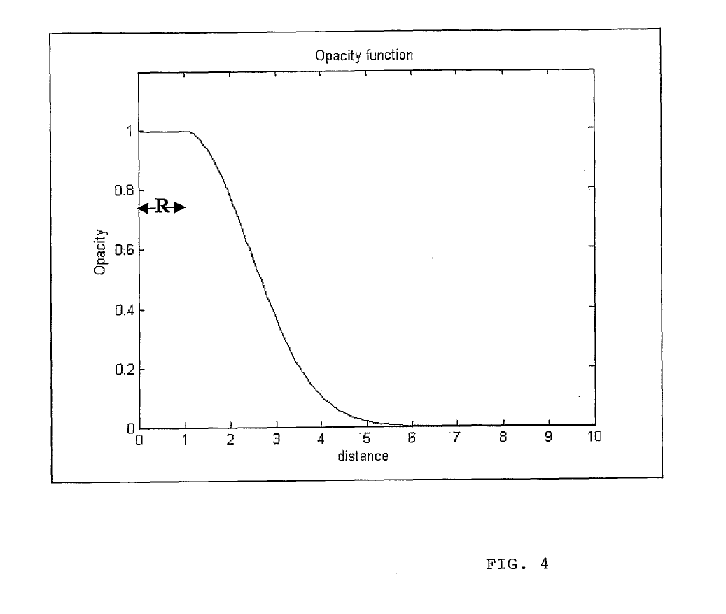

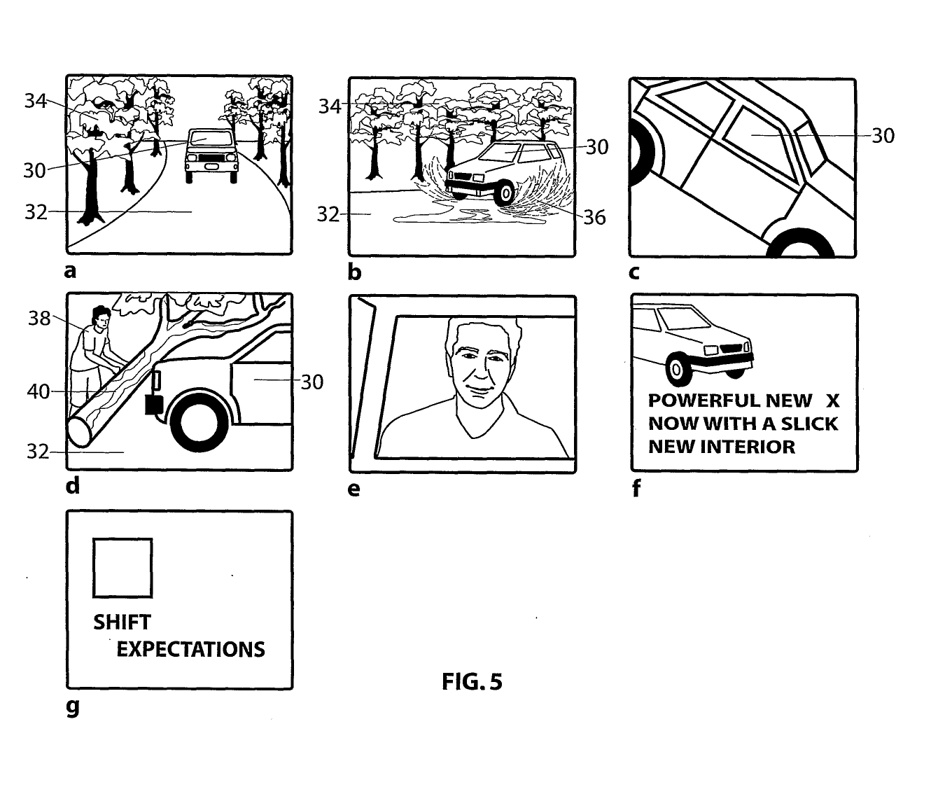

FIG. 5 represents still frames from a video advertisement; and

FIGS. 6 to 11 illustrate data obtained from subjects as a function of time.

FIG. 1 schematically illustrates a system for determining the response of a subject or a group of subjects to audiovisual material presented on a video screen 3 and loudspeaker 2. The system includes a computer 1 which controls various parts of the hardware and also performs computation on signals derived from the brain activity of the subject 7, as will be described below. The computer 1 also holds the television program and advertisements which can be presented to one or more subjects 7 on the screen 3 and/or through the loudspeaker 2.

The subject or subjects 7 to be tested are fitted with a headset 5 which includes a plurality of electrodes for obtaining brain electrical activity from various sights on the scalp of the subject 7. The headset includes a visor 4 which has for the left and right eyes of the subject half silvered mirrors 8 and white light Light Emitting Diode (LED) arrays 9, as shown in FIG. 2. The half silvered mirrors 8 are arranged to direct light from the LED arrays 9 towards the eyes of the subject 7. The LED arrays 9 are controlled so that the light intensity therefrom varies sinusoidally as a function of time; under the control of control circuitry 6. The control circuitry 6 includes a waveform generator for generating the sinusoidal signal. The circuitry 6 also includes amplifiers, filters, analogue to digital converters and a Universal Serial Bus (USB) interface or a Transmission Control Protocol (TCP) interface or other digital interface for coupling the various electrode signals into the computer 1, as will be described in more detail below.

A translucent screen 10 is located in front of each LED array 9. Printed on the screen is an opaque pattern. The opacity of the opaque pattern is a maximum in a circular area in the centre of the centre of the screen. Beyond the circular area, the opacity falls off smoothly with radial distance from the circular area circumference, preferably, the opacity falls off as a Gaussian function described by Equation 1. The screen therefore reduces the flicker in the central visual field thus giving subjects a clear view of the visually presented material. The size of the central opaque circle (not shown) should be such as to occlude the visual flicker in the central visual field between 4°-6° vertically and horizontally. In other words the screen 10 blocks the flicker from the fovea of the subjects. The screen 3 presenting the advertisements typically subtends an angle of 10°-14° vertically and horizontally as measured from the eyes of the subject.

If r<R then P=1

If r≧R then P is given by the equation 1 below.

P=e^{-(r-R)}^{²}^{/G}^{²} Equation 1

where P is the opacity of the pattern on the translucent screen 10

An opacity of P=1.0 corresponds to no light being transmitted through the screen while an opacity of P=0 corresponds to complete transparency.

R is the radius of the central opaque disk while r is the radial distance from the centre of the opaque disk. G is a parameter that determines the rate of fall-off of opacity with radial distance. Typically G has values between R/4 and 2R. FIG. 4 illustrates the fall-off of opacity with radial distance from the centre of the disk. In FIGS. 4, R=1 and G=2R. While a Gaussian fall-off of opacity with radius is preferable, any function that is smooth and has a zero gradient at r=R and at r>3G will be suitable.

The computer 1 includes software which calculates SSVEP amplitude phase and/or coherence from each of the electrodes in the headset 5. Details of the hardware and software required for generating SSVEP are well known and need not be described in detail. In this respect reference is made to the aforementioned U.S. patent specifications which disclose details of the hardware and techniques for computation of SSVEP. Briefly, the subject 7 views the video screen 3 through the special visor 4 which delivers a background flicker to the peripheral vision. The frequency of the background flicker is typically 13 Hz but may be selected to be between 3 Hz and 50 Hz. More than one flicker frequency can be presented simultaneously. The number of frequencies can vary between 1 and 5. Brain electrical activity will be recorded using specialized electronic hardware that filters and amplifies the signal, digitizes it in the circuitry 6 where it is then transferred to the computer 1 for storage and analysis. SSPT is also used to ascertain regional brain activity at the scalp sites using SSPT analysis software, which is known and does not need to be described herein.

As mentioned above, the visor 4 includes LED arrays 9. In one embodiment, the light therefrom is varied sinusoidally. An alternative approach utilises pulse width modulation where the light emitting sources are driven by 1-10 Khz pulses and the pulse duration is proportional to the brightness of the light emitting sources. In this embodiment, the control circuitry 6 receives a digital input stream from the computer 1 and outputs pulse width modulated pulses at a frequency of 1-10 Khz. The time of each positive going zero-crossing from the sinusoidal stimulus waveform or combination of stimulus waveforms is preferably determined to an accuracy of about 10 microseconds and stored in the memory of the computer 1.

Brain electrical activity is recorded using outputs from the multiple electrodes in headset 5 or another commercially available multi-electrode system such as Electro-cap (ECI Inc., Eaton, Ohio USA). The number of electrodes is normally not less than 8 and normally not more than 128, typically 16 to 32. The electrodes are disposed so as to obtain outputs from selected scalp sites which are identified by the International 10-20 System of electrode locations shown schematically in FIG. 3.

Brain activity at each of the electrodes is conducted to the control circuitry 6. The circuitry 6 includes multistage fixed gain amplification, band pass filtering and sample-and-hold circuitry for each channel. Amplified/filtered brain activity is preferably digitized to 16-24 bit accuracy at a rate not less than 300 Hz and transferred to the computer 1 for storage on hard disk (not shown). The timing of each brain electrical sample together with the time of presentation of different features of the audiovisual material are also registered and preferably stored to an accuracy of 10 microseconds. The details of the control circuitry 6 are well known and need not be described.

### SSVEP Amplitude and Phase

The digitized brain electrical activity (electroencephalogram or EEG) together with timing of the stimulus zero crossings enables one to calculate the SSVEP elicited by the flicker at a particular stimulus frequency from the recorded EEG or from EEG data that has been pre-processed using Independent Components Analysis (ICA) to remove artefacts and increase the signal to noise ratio. ICA is a mathematical technique for decomposing a signal into independent constituent components. This can be used to remove artefacts because artefact signals will be independent of brain activity. Bell A. J. and Sejnowski T. J. 1995, An information maximisation approach to blind separation and blind deconvolution, Neural Computation, 7, 6, 1129-1159; T-P. Jung, S. Makeig, M Westerfield, J. Townsend, E. Courchesne and T. J. Sejnowskik, Independent component analysis of single-trial event-related potential Human Brain Mapping, 14(3):168-85,2001.

### Calculation of SSVEP Amplitude and Phase

Calculation of SSVEP amplitude and phase coefficients for each stimulus cycle for a given stimulus frequency can be accomplished used Fourier techniques using Equations 2 and 3 below.

$${a}_{n} = \frac{1}{S Δ τ} {\sum }_{i = 0}^{S - 1} f ( nT + i Δ τ ) cos ( \frac{2 π}{T} ( nT + i Δ τ ) ) \\ {b}_{n} = \frac{1}{S Δ τ} {\sum }_{i = 0}^{S - 1} f ( nT + i Δ τ ) sin ( \frac{2 π}{T} ( nT + i Δ τ ) ) \qquad Equation 2$$

Equation 2 describes the calculation of stimulus frequency Fourier coefficients for a single cycle of the flicker stimulus. Essentially, the EEG output is sampled at specific points in time and is represented by f(nT+iΔτ) while the stimulus is represented by the cosine function in Equation 2 and the stimulus waveform shifted by 90 degrees is represented by the sine function. Equation 2 enables calculation of the single cycle Fourier coefficients (real and imaginary) by calculating the sum of the product of the EEG output waveform and the stimulus waveform (cos) and the stun of the product of the EEG output waveform and the stimulus waveform phase shifted by 90 degrees (sin) over a single cycle. The single cycle Fourier coefficients are smoothed by averaging overlapping sets of Fourier coefficients, where:

aₙ and bₙ are the cosine and sine Fourier coefficients respectively where;

n represents the nth stimulus cycle;

S is the number of samples per stimulus cycle (16);

Δτ is the time interval between samples;

T is the period of one cycle; and

f(nT+iΔτ) is the EEG signal (raw or pre-processed using ICA).

SSVEP_{amplitude} and SSVEPₚₕₐₛₑ can be calculated using Equation 3 below.

$${SSVEP}_{amplitude} = \sqrt{( {A}_{n}^{2} + {B}_{n}^{2} )} \\ {SSVEP}_{phrase} = a tan ( \frac{{B}_{n}}{{A}_{n}} ) \qquad Equation 3$$

Where Aₙ and Bₙ are overlapping smoothed Fourier coefficients calculated by using Equation 4 below.

$${A}_{n} = {\sum }_{i = 1}^{i = N} {a}_{n + i} / N \\ {B}_{n} = {\sum }_{i = 1}^{i = N} {b}_{n + i} / N \qquad Equation 4$$

Amplitude and phase components can be calculated using either single cycle Fourier coefficients (aₙ and bₙ) or coefficients that have been calculated by smoothing across multiple cycles (Aₙ and Bₙ).

Equations 3 and 4 describe the procedure for calculating the smoothed SSVEP coefficients for a single subject. For pooled data, the SSVEP coefficients (Aₙ and Bₙ) for a given electrode are averaged (or pooled) across all of the subjects or a selected group of subjects.

As the number of cycles used in the smoothing increases, the signal to noise ratio increases while the temporal resolution decreases. The number of cycles used in the smoothing is typically in excess of 5 and less than 130.

The above equations apply to scalp recorded data as well as brain electrical activity inferred at the cortical surface adjacent to the skull and deeper regions. Activity in deeper regions of the brain such as the orbito-frontal cortex or ventro-medial cortex can be determined using a number of available inverse mapping techniques such as BESA, EMSE and LORETA.

Once the system has been set up and headsets 5 fitted to one or more subjects, the audiovisual program or advertisement to be evaluated is displayed on the screen 3, the visual flicker is switched on in the visor 4 and brain electrical activity of the subject or subjects is recorded continuously on the computer 1. At the end of the recording stage, the SSVEP amplitude and phase are separately calculated for each subject. Once all recordings are completed, group averaged data is calculated by averaging the smoothed SSVEP amplitude and phase data from selected subjects to be included in the group (eg male, female, young, old). Changes in regional synaptic excitation or inhibition are indicated by SSVEP phase changes while changes in regional activity (irrespective of whether these changes are associated with excitation or inhibition) are indicated by changes in SSVEP amplitude. Typically, such inverse mapping techniques require 19 or more scalp recording sites and preferably 64 or more scalp recording sites.

In accordance with the invention, cognitive and emotional measures at specific points in time during the advertisement can be derived from the pattern of SSVEP phase and SSVEP amplitude changes at the various scalp recording sites or inferred brain regional activity. Scalp recording sites typically used are identified by reference to the International 10-20 System (Jasper H H: Electroenceph. Clin. Neurophysiol. 1958; 10: 370-1) shown in FIG. 3. The psychological states associated with SSVEP phase and amplitude changes at scalp and brain cortical locations are summarised below.

Activity at various brain sites can be determined using a variety of possible inverse mapping techniques such as EMSE and LORETA that express brain activity in selected brain regions in terms of a function of SSVEP amplitude and phase measures recorded over the entire scalp. Typically such techniques require more than 20 scalp recording sites and preferably 64 or more scalp recording sites.

### 1. Visual Attention to Detail

This measure refers to the level of visual attention to detail. Examples include visual attention to text and numbers as well as visual attention to details of a scene or a face.

SSVEP phase advance or amplitude change at the left occipital region, preferably electrode O₁ has been found to be relevant to assessment of the subject's Visual Attention to Detail. If inverse mapping techniques are used, the relevant location in the left cerebral cortex is the vicinity of Brodmans area 17.

### 2. Visual Attention to Global Features

This measure refers to the level of visual attention to global features. Examples include visual attention to images such as scenery or faces as a whole.

SSVEP phase advance or amplitude change at the right occipital region, preferably electrode O₂ has been found to be relevant to the assessment of the subject's Visual Attention to Global Features including responses to facial expressions displayed on the screen. If inverse mapping techniques are used, the relevant location in the right cerebral cortex is the vicinity of Brodmans area 17.

### 3. Multi-Modal Attention to Detail or Desirability

This measure refers to multi-modal or multi-sensory attention. Typically this includes both auditory and visual attention to detail in the visual domain or speech in the auditory domain. When this attention measure is also associated with objects, it indexes the level of desirability associated with the said object.

SSVEP phase advance or amplitude change at the left parietal region, preferably electrode P₃ has been found to be relevant to the assessment of the subject's Multi-modal Attention to Detail or Desire for the subject matter of the screened advertisement. If inverse mapping techniques are used, the relevant location in the left cerebral cortex is the vicinity of the left intraparietal area.

### 4. Multi-Modal Attention to Global Features or Desirability

This measure refers to multi-modal or multi-sensory attention. Typically this includes both auditory and visual attention to global features such as facial expression, scenery in the visual domain or music in the auditory domain. When this attention measure is also associated with objects, it indexes the level of desirability associated with the said object.

SSVEP phase advance or amplitude change at the right parietal region, preferably electrode P₄ has been found to be relevant to the assessment of the subject's Multi-modal Attention to Global Features or Desire for the subject matter of the screened advertisement. If inverse mapping techniques are used, the relevant location in the left cerebral cortex is the vicinity of the right intraparietal area.

### 5. Emotional Intensity

This measure indicates the intensity of the emotional state experienced by subjects. This measure is independent of the specific emotion such as joy, fear, anger, anxiety, etc.

SSVEP phase advance or amplitude change at the right parieto-temporal region, preferably from a single electrode which is approximately equidistant from right hemisphere electrodes O₂, P₄ and T₆, has been found to be relevant to the assessment of the subject's Emotional Intensity response to different parts of the screened advertisement. If inverse mapping techniques are used, the relevant location in the right cerebral cortex is the vicinity of the right parieto-temporal junction.

### 6. Attraction-Repulsion (Sometimes Termed Like-Dislike)

This measure indicates the extent to which subjects are attracted or repelled to characters or situations represented in the advertisements. When activity at the combination of left hemisphere sites is greater than at the combination of right hemisphere sites, subjects are attracted to the scene or character and vice versa.

The difference between SSVEP phase advance at the left frontal/prefrontal and right frontal/prefrontal regions has been found to be relevant to the assessment of the subject's Attraction-Repulsion response to the screened advertisement. Attraction is indicated by a larger phase advance in the left hemisphere compared to the right while Repulsion is indicated by a larger phase advance in the right hemisphere compared to the left. Attraction-Repulsion can be calculated as follows:

Attraction=(a₁*SSVEP phase advance at electrode F₃+a₂*SSVEP phase advance at electrode Fₚ₁−a₃*SSVEP phase advance at electrode F₄−a₄*SSVEP phase advance at electrode Fₚ₂)

where a₁=a₂=a₃=a₄=1.0 Equation 5

A positive value for the Attraction measure is associated with the subjects finding the screened material attractive and liked while a negative measure for Attraction is associated with repulsion or dislike of the screened material.

If inverse mapping techniques are used, the Attraction can be calculated as follows:

Attraction=(c₁*right orbito-frontal cortex (in vicinity of Brodman area 11)+c₂*right dorso-lateral prefrontal cortex (in vicinity of Brodman area 9)+c₃*left orbito frontal cortex (in vicinity of Brodman area 11)+c₄*left dorso-lateral prefrontal cortex (vicinity of Brodman area 9))

where c₁=1, c₂=1, c₃=1, c₄=1 Equation 6

and wherein the averaged SSVEP phase measures at the above sites are inserted in Equation 6.

### 7. Engagement

This measure indicates the level of Engagement. Engagement refers to the extent that different components of the advertisement elicit a sense of personal relevance.

Engagement of the subject in the screened advertisement can be calculated by the weighted mean SSVEP phase advance at prefrontal sites described by the expression below.

Engagement=(b₁*SSVEP phase advance at electrode F₃+b₂*SSVEP phase advance at electrode Pₚ₁+b₃*SSVEP phase advance at electrode F₄+b₄*SSVEP phase advance at Electrode Fₚ₂)

where b₁=0.1, b₂=0.4, b₃=0.1, b₄=0.4 Equation 7

If inverse mapping techniques are used, the relevant expression is:

Engagement=(d₁*right orbito frontal cortex (in vicinity of Brodman area 11)+d₂*right dorso-lateral prefrontal cortex (in vicinity of Brodman area 9)+d₃*left orbito frontal cortex (in vicinity of Brodman area 11)+d₄*left dorso-lateral prefrontal cortex (in vicinity of Brodman area 9))

where d₁=0.1, d₂=0.4, d₃=0.1, d₄=0.4 Equation 8

and wherein the averaged SSVEP phase measures at the above sites are inserted in Equation 8.

### 8. Behavioural Intent

Behavioural Intent or the likelihood that the subjects are likely to act in accordance with the purpose of the screened advertisement, that is make a purchase, is associated with memory encoding at the time of branding. This is indicated by the level of long term memory encoding at the time that the brand is portrayed in the advertisement. Long term memory encoding can be assessed as follows:
SSVEP phase advance or amplitude change at left frontal region, preferably a single electrode which is approximately equidistant from left hemisphere electrodes C₃, F₃ and F₇, has been found to be relevant to the assessment of the subject's long term memory encoding for details and verbal memories of the subject matter of the screened advertisement. If inverse mapping techniques are used, the relevant location in the left cerebral cortex is the vicinity of Brodmans areas 6, 44, 45, 46 and 47.
SSVEP phase advance or amplitude change at right frontal region, preferably approximately equidistant from right hemisphere electrodes C₄, F4 and F₈ has been found to be relevant to the assessment of the subject's long term memory encoding for emotional and non-verbal memories of the subject matter of the screened advertisement. If inverse mapping techniques are used, the relevant location in the right cerebral cortex is the vicinity of Brodmans areas 6, 44, 45, 46 and 47.

Once data from long term memory encoding has been obtained for verbal and/or non-verbal memories, this can be correlated with the time sequence of the advertising material being presented to determine the relevant levels of memory encoding when the brand is portrayed in the advertisement. After this correlation, it is then possible to assess whether or not the subjects are likely to purchase goods or services shown in the advertisement.

### Example

The following procedure is used to evaluate a television advertisement to a client such as the brand manager of a company and/or advertising agency.

A selected number of subjects, say 50, are seated in a test room and the headsets 5 are placed on their heads. The visors 4 are then placed in position and adjusted so that the foveal block by the screens 10 prevents the appearance of the flicker over the screens 3 where the advertisements are presented. The number of subjects in a recording session is variable and typically can vary from 1 to over 100. When pooling subjects to create the average response, the number of subjects whose data is to be included in the average should be no less than 16.

The advertisements to be tested were included in an ‘advertising break’ comprising a block of 3 to 6 advertisements incorporated in a television program to simulate a standard commercial TV viewing environment. Each advertising break is followed immediately by a 30 second sequence of still images of scenery and a musical accompaniment. Typically, 60 images were presented over the period of 30 seconds with each image present for 0.5 seconds. The same sequence of images and music were presented after each advertisement break. Brain activity levels during the adjacent scene images are used as a reference level for brain activity during the preceding advertisement break. This enables removal of any long-term changes in brain activity that may occur over the time course of the recording period.

Pooled or averaged data at various brain sites can then be displayed to the client as the difference between the reference level and the value at other points in time during the advertisement. A fixed offset between 0.2 to 0.6 preferably 0.3 radians is then added to the abovementioned difference to yield the SSVEP phase data at each scalp site.

To minimize subject irritation or discomfort to the subject due to the flicker, the flicker stimulus is of variable intensity and only switched to the highest intensity when material of interest to the client such as the block of advertisements is present on the screen. During the periods that material of interest is not present on the screen, the stimulus intensity is typically zero and never more than 10% of the typical value used when material of interest is on the screen. Preferably, the stimulus is not switched on abruptly but is slowly increased before the advertisement break and decreased slowly after the end of an advertisement break. Typically, the stimulus is increased linearly over a 30-60 second epoch prior to the advertisement break so that it reaches its maximum value 60 seconds prior to the first advertisement. Immediately the sequence of reference images at the end of the advertisement break ends, the stimulus intensity is linearly reduced to the minimum value over a 30 second period. The slow linear increase and decrease of stimulus intensity occurs for every advertisement break.

FIG. 5 schematically illustrates the television advertisement to be assessed. The advertisement is in relation to a sports utility vehicle 30. The vehicle 30 is being driven along a road 32 in a dark forest 34 on a wet and stormy night, as indicated by still segment a. The vehicle 30 drives through a partially flooded roadway so as to cause a large spray 36 from the vehicle, as indicated in still segment b. The vehicle is then shown traversing steep grade, as shown in still segment c. The driver 38 then stops the vehicle where a large log 40 has fallen on the road 32 so as to remove it, as shown in still segment d. After clearing the branch from the road, the driver is about to re-enter the car when he realises that he is covered with mud and would soil the interior of the car were he to enter immediately, as indicated in still segment e. This scene features an abrupt change in the soundtrack when the music stops and is replaced by a period of silence. The driver ruefully finds a cake of soap in the glove compartment and proceeds to take a bath in the nearby stream during the storm. The final segment of the animated part of the advertisement shows a block of text with the brand of the vehicle in the text block, as indicated by still segment f. The final segment g runs for about 2 seconds and shows the brand and product being advertised.

Once the final subject recording has taken place, individual SSVEP data is pooled for each specific group of subjects, for example, separate pooling or averaging could be effected for the entire group and also for subgroups such as male and female and young and old. To display the SSVEP phase data in relationship to the associated commercial, an in-house program (NV-Show) is used. NV-Show displays the advertisement in a portion of a computer video monitor and the associated SSVEP phase data as a graph below. As the advertisement is played in the upper portion of the screen, the graph describing the SSVEP phase data is revealed below. Alternatively, the graph of SSVEP phase data can be present throughout the advertisement playing time and a moving time marker used to indicate the SSVEP phase data corresponding to time in the advertisement being displayed.

FIGS. 6 to 11 illustrate the brain measurement values at the various brain sites during the advertisement. The block arrows labelled ‘a’ to ‘g’ in FIGS. 6 to 11 correspond to the points in time a to g shown in FIG. 5.

In FIG. 6, the line 50 shows the Attraction-Repulsion measure during the advertisement. The Attraction-Repulsion measures are calculated using Equation 5 above and the results are displayed in graphical form on a time scale which corresponds to the duration of the advertisement. Positive values or values above the horizontal time axis correspond to ‘approach’ or ‘like’ while negative values correspond to points in time where the audience ‘withdraws’ or ‘dislikes’. For the approach-withdraw measure in this study, any value larger than =0.3 or smaller than −0.3 is statistically significant.

If a greater number of electrodes were used, it would be possible to use inverse mapping techniques to obtain somewhat higher resolution in the data and by using Equation 5.

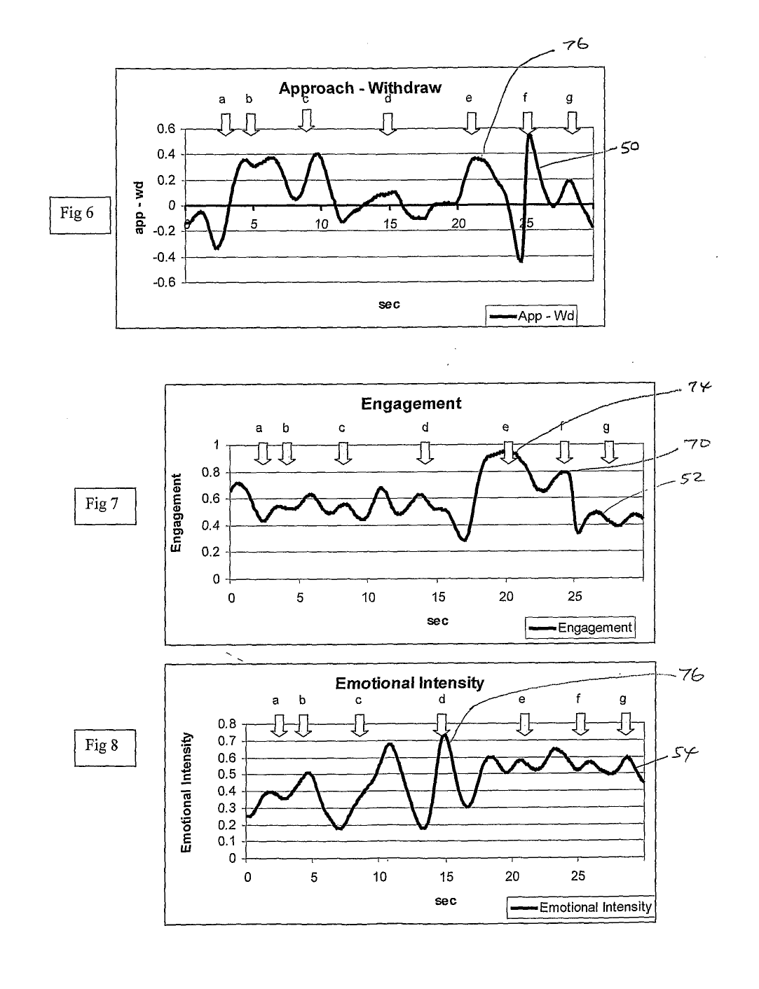

In FIG. 7 the line 52 indicates the level of audience Engagement in the advertisement as a function of time. The data is calculated using Equation 7 and is again presented graphically on a time scale which corresponds to that of the advertisement. The term Engagement relates to the level of personal or emotional relevance experienced by the subjects. In the study illustrated, Engagement must exceed 0.4 to be significantly greater than baseline level while an engagement level above 0.7 is considered high.

If inverse mapping techniques are used, Equation 8 can be used to calculate the measure for Engagement.

In FIG. 8 the line 54 indicates the equivalent changes in Emotional Intensity elicited by the advertisement. Emotional Intensity refers to the intensity of emotion experienced by the subjects irrespective of the type of emotion, e.g. fear, joy, anger, excitement etc. The data can be calculated as described above by using SSVEP phase advance or amplitude change at right parieto-temporal region, from an electrode which is approximately equidistant from the right hemisphere electrodes O₂, P₄ and T₆. If inverse mapping techniques are used, the relevant location in the right cerebral cortex is in the vicinity of the right parieto-temporal junction. Once again, any level above 0.4 is significantly higher than background while a level above 0.7 is considered high.

The lines 56 and 58 in FIG. 9 indicate memory encoding in detail and memory encoding global respectively during the advertisement. This measure indicates how effectively different components of the advertisement are being stored in long term memory. This measure is especially important as an advertisement is only effective if the key messages and information about the brand enters long-term memory. It has been found that the likely impact of the advertisement on Behavioural Intent is positively correlated with the level of long-term memory encoding during the time that branding information is presented in the advertisement. Thus Behavioural Intent or the likely impact on behaviour of the subjects is indicated by the level of memory encoding at the time that branding information is presented in the advertisement, i.e. at points f and g where the branding information is positively displayed during the advertisement. Both memory traces are important in regard to Behavioural Intent. As long as either left or right memory encoding state is high during “branding”, Behavioural Intent or the propensity to act on the message of the advertisement, is high.

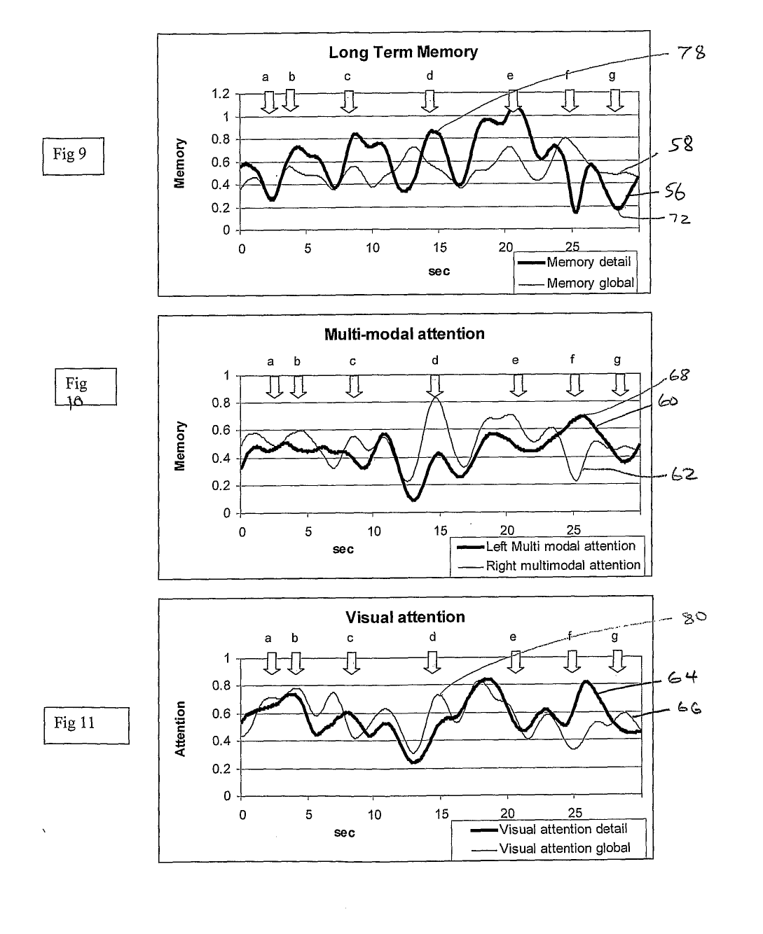

In FIG. 10 the lines 60 and 62 indicate left hemisphere multi-modal attention and right hemisphere multi-modal attention respectively. Multi-modal attention includes visual and auditory attention to details of the advertisement being displayed to the subjects. Specifically, the line 60 indicates attention to detailed features such as text and speech and the line 62 indicates attention to global features such as facial expression and music (for example).

In FIG. 11 the lines 64 and 66 indicate the visual attention to detail and visual attention globally of the subjects to the advertising material being displayed. Specifically, the line 64 indicates visual attention to detailed features such as text. The line 64 indicates visual attention to global features such as facial expression (for example).

### Assessment

For this advertisement to be assessed as effective, it is necessary for memory encoding to be high during the times when the product or product benefits are emphasized, such as at point f and when the brand is explicitly featured such as at point g. As can be seen in FIG. 10, left memory encoding is moderate to high during point f as indicated by peak 68 in the line 60 indicating that there is adequate memory encoding for the text information THE POWERFUL NEW X; NOW WITH A SLICK NEW INTERIOR near the end of the advertisement. During this point in time, Engagement is also high indicating a high degree of personal relevance regarding the features mentioned as indicated by the peak 70 in line 52.

By contrast, the memory encoding for the branding information at point g is low and Engagement is also low for this time as indicated by trough 72 in the line 56. This indicates that the subjects will remember the vehicle interior but will not recall the brand. The Behavioural Intent measure is also low indicating that the advertisement is not effective as the subjects will be less inclined to act in accordance with the advertisement. This is again indicated by the trough 72.

Accordingly, the advertisement can be assessed as not being commercially effective in that the branding information is not satisfactorily encoded in long-term memory. The dramatic structure of the advertisement is, however, effective in that ‘joke’ at point e in the advertisement works because the scene where the driver realizes he can't re-enter the car without washing is well encoded in long-term memory and also leads to highest level of Engagement as indicated by the peak 74 in line 52. In addition, there is a high level of Attraction that is associated with this humour as indicated by peak 76 in line 50.

It is also apparent that the overall structure of the advertisement is effective in that the key dramatic scene where the driver has to get out of the car to clear the road at point d is associated with high levels of Emotional Intensity as indicated by the peak 76 in line 54; high levels of memory encoding as indicated by peak 78 in line 56; and a high level of Attention to Global Features as indicated by the peak 80 in line 66.

After assessment of the advertisement using the techniques of the invention, the client can appreciate that the overall effect of the advertisement is positive but some modification is required at points f and g where the brand is introduced. Accordingly, the client is in a position to modify the advertisement so that it should become highly successful and avoid incurring substantial advertising costs in an advertising program which does not induce viewers to purchase the product.

It will be appreciated that the principles of the invention can be applied other audiovisual commercial communications in addition to advertisements.

Many modifications will be apparent to those skilled in the art without departing from the spirit and scope of the invention.

1. A method of quantitatively assessing the effectiveness of an audiovisual, visual or audio advertisement including the steps of:
presenting the advertisement to a plurality of subjects, the advertisement having a sequence of audiovisual, visual and/or audio features which occur as a function of time;
obtaining, during presentation of the advertisement, EEG signals from the subjects from predetermined scalp sites thereof;
calculating SSVEP amplitudes and/or phase differences from EEG signals obtained from said predetermined scalp sites in order to obtain output signals which represent predetermined psychological states of each subject to said features as a function of time;
combining the output signals from said subjects to obtain pooled output signals; and
displaying the pooled output signals to thereby enable quantitative assessment of the subjects' responses to said features of the advertisement in order to assess the effectiveness of the features of the advertisement.

2. A method as claimed in claim 1 including the step of simultaneously displaying the advertisement to said plurality of subjects.

3. A method as claimed in claim 1 wherein the step of combining the outputs includes the step of averaging output signals from each subject.

4. A method as claimed in claim 2 including the step of selecting scalp sites in order to obtain output signals which enable assessment of:
visual attention to detail;
visual attention to global features;
multi-modal attention to detail or desirability;
multi-modal attention to global features or desirability;
emotional intensity;
attraction-repulsion;
engagement; or
behavioural intent
in relation to the features of the advertisement.

5. A method as claimed in claim 4 including the step of applying a sinusoidally varying visual flicker stimulus to each subject during presentation of the advertisement to thereby enable calculation of Fourier coefficients from said output signals to thereby enable calculation of said SSVEP amplitudes and/or phase differences.

6. A method as claimed in claim 5 wherein said SSVEP amplitude and phase are calculated by the equations:
$${SSVEP}_{amplitude} = \sqrt{( {A}_{n}^{2} + {B}_{n}^{2} )} \\ {SSVEP}_{phrase} = a tan ( \frac{{B}_{n}}{{A}_{n}} ) \qquad$$
where: aₙ and bₙ are cosine and sine Fourier coefficients calculated by the equations:
$${a}_{n} = \frac{1}{S Δ τ} {\sum }_{i = 0}^{S - 1} f ( nT + i Δ τ ) cos ( \frac{2 π}{T} ( nT + i Δ τ ) )$$
$${b}_{n} = \frac{1}{S Δ τ} {\sum }_{i = 0}^{S - 1} f ( nT + i Δ τ ) sin ( \frac{2 π}{T} ( nT + i Δ τ ) )$$
where:
aₙ and bₙ are the cosine and sine Fourier coefficients respectively where;
n represents the nth flicker stimulus cycle;
S is the number of samples per flicker stimulus cycle;
Δτ is the time interval between samples;
T is the period of one cycle;
f(nT+iΔτ) is the EEG signal (raw or pre-processed using ICA) obtained from said predetermined scalp sites;
and wherein Aₙ and Bₙ are overlapping smoothed Fourier coefficients calculated by using the equation:
$${A}_{n} = {\sum }_{i = 1}^{i = N} {a}_{n + i} / N$$
$${B}_{n} = {\sum }_{i = 1}^{i = N} {b}_{n + i} / N$$

7. A method as claimed in claim 6 including the steps of:
obtaining EEG signals from a plurality of scalp sites of each subject; and
utilising inverse mapping techniques such as BESA, EMSA or LORETA to produce modified EEG signals which represent activity in deeper regions of the brain of each subject such as the orbito-frontal cortex or the ventro-medial cortex.

8. A method as claimed in claim 6 including the step of averaging the Fourier coefficients Aₙ and Bₙ for a selected group of subjects and then calculating the SSVEP amplitudes and SSVEP phase differences for said group of subject.

9. A method as claimed in claim 5 wherein the flicker signal is applied only to the peripheral vision of each subject.

10. A method as claimed in claim 9 including the steps of directing the light towards the eyes of each subject via first and second screens so that the light passing through the screen constitutes said flicker signal and wherein each screen includes an opaque area, and wherein the method further includes the step of positioning the screens to the relative position of each subject such that said opaque areas prevent said flicker signal impinging on the fovea of each eye of each subject.

11. A method as claimed in claim 10 wherein the opacity of each screen decreases as a function of distance from its opaque area so that the intensity of the flicker signal impinging on each retina of each subject decreases in value from the central vision to the peripheral vision.

12. A method as claimed in claim 11 including the step of applying a masking pattern to each screen to define the opacity thereof, the method including the step of applying the pattern in accordance with a masking pattern function which provides zero or low gradients for changes in opacity adjacent to its opaque area and peripheral areas thereof which define parts of the flicker signal impinging on the peripheral vision of each subject.

13. A method as claimed in claim 12 wherein the opaque area of each screen is circular and wherein the masking pattern function is selected to be a Gaussian function, so that the opacity P of the screen is defined by the equation:
P=e^{-(r-R)}^{²}^{/G}^{²}
where:
r is the radial distance from the centre of the opaque area; and
G is a parameter that determines the rate of fall-off of opacity with radial distance, and wherein when r<R, P=1.

14. A method as claimed in claim 13 wherein G has a value in the range R/4 and 2R.

15. A method as claimed in claim 6 including the steps of applying an electrode to the scalp of each subject at the O₁ site, calculating SSVEP amplitudes and phase differences from EEG signals from said electrode whereby the output signals indicate each subject's visual attention to details of the advertisement.

16. A method as claimed in claim 7 including the step of utilising inverse mapping determines brain activity in the left cerebral cortex in the vicinity of Brodman's area 17 whereby the modified output signals indicate each subject's visual attention to details of the advertisement.

17. A method as claimed in claim 6 including the steps of applying an electrode to the scalp of each subject at the O₂ site, calculating SSVEP amplitudes and phase differences from EEG signals from said electrode whereby the output signals are indicative of each subject's visual attention to global features of the advertisement.

18. A method as claimed in claim 7 including the step of utilising inverse mapping determines brain activity in the right cerebral cortex in the vicinity of Brodman's area 17 whereby the output signals indicate each subject's visual attention to global features of the advertisement.

19. A method as claimed in claim 6 including the step of applying an electrode to the scalp of each subject at the P₃ site, calculating SSVEP amplitudes and phase differences from EEG signals from said electrode whereby the output signals are indicative of each subject's multi-modal attention to detail or desirability to features of the advertisement.

20. A method as claimed in claim 7 wherein the step of utilising inverse mapping determines brain activity in the left cerebral cortex in the vicinity of the intraparietal area whereby the output signals indicate each subject's multi-modal attention to detail or desirability of the features of the advertisement.

21. A method as claimed in claim 6 including the step of applying an electrode to the scalp of each subject at the P₄ site, calculating SSVEP amplitudes and phase differences from EEG signals from said electrode whereby the output signals indicate each subject's multi-modal attention to global features or desirability of the features of the advertisement.

22. A method as claimed in claim 7 wherein the step of utilising inverse mapping determines brain activity in the right cerebral cortex in the vicinity of the intraparietal area whereby the output signals indicate each subject's multi-modal attention to global features or desirability of the features of the advertisement.

23. A method as claimed in claim 6 including the step of applying an electrode to the scalp of each subject at a site which is approximately equidistant from sites O₂, P₄ and T₆, calculating SSVEP amplitudes and phase differences from EEG signals from said electrode whereby the output signals indicate each subject's emotional intensity associated with the advertisement.

24. A method as claimed in claim 7 wherein the step of utilising inverse mapping determines brain activity in the right cerebral cortex in the vicinity of the right parieto-temporal junction whereby the output signals indicate each subject's emotional intensity associated with the advertisement.

25. A method as claimed in claim 6 including the steps of applying an electrode to the scalp of each subject at the F₃, F₄, Fₚ₁ and Fₚ₂ sites, calculating SSVEP amplitudes and phase differences from EEG signals from said electrodes, calculating values for attraction-repulsion using the equation:
attraction=(a₁*SSVEP phase advance at electrode F₃+a₂*SSVEP phase advance at electrode Fₚᵢ−a₃*SSVEP phase advance at electrode F₄−a₄*SSVEP phase advance at electrode Fₚ₂)
where a₁=a₂=a₃=a₄=1.0
whereby said values indicate each subject's attraction or repulsion towards features of the advertisement.

26. A method as claimed in claim 7 wherein the step of utilising inverse mapping determines brain activity in:
the right orbito-frontal cortex in the vicinity of Brodman area 11;
the right dorso-lateral prefrontal cortex in the vicinity of Brodman area 9;
the left orbito frontal cortex in the vicinity of Brodman area 11; and
the left dorso-lateral prefrontal cortex in the vicinity of Brodman area 9; and
calculating a value for attraction-repulsion using the equation:
attraction=(c₁*right orbito-frontal cortex (in vicinity of Brodman area 11)+c₂*right dorso-lateral prefrontal cortex (in vicinity of Brodman area 9)+c₃*left orbito frontal cortex (in vicinity of Brodman area 11)+c₄*left dorso-lateral prefrontal cortex (vicinity of Brodman area 9))
where c₁=1, c₂=1, c₃=1, c₄=1,
whereby said values indicate each subject's attraction or repulsion towards features of the advertisement.

27. A method as claimed in claim 6 including the steps of applying electrodes to the scalp of each subject at F₃, F₄, Pₚ₁ and Fₚ₂ sites, calculating SSVEP amplitudes and phase differences from said electrodes, calculating values for engagement in features of the advertisement by a weighted mean SSVEP phase advance at said sites using the equation:
engagement=(b₁*SSVEP phase advance at electrode F₃+b₂*SSVEP phase advance at electrode Pₚ₁+b₃*SSVEP phase advance at electrode F₄+b₄*SSVEP phase advance at Electrode Fₚ₂)
where b₁=0.1, b₂=0.4, b₃=0.1, b₄=0.4,
whereby said values indicate each subject's engagement in features of the advertisement.

28. A method as claimed in claim 7 wherein the step of utilising inverse mapping determines brain activity in:
the right orbito frontal cortex in the vicinity of Brodman area 11;
the right dorso-lateral prefrontal cortex in the vicinity of Brodman area 9;
the left frontal cortex in the vicinity of Brodman area 11; and
the left dorso-lateral prefrontal cortex in the vicinity of Brodman area 9,
calculating SSVEP amplitudes and phase differences from said modified EEG signals from said electrodes; and
calculating a value for engagement using the equation:
engagement=(d₁*right orbito frontal cortex (in vicinity of Brodman area 11)+d₂*right dorso-lateral prefrontal cortex (in vicinity of Brodman area 9)+d₃*left orbito frontal cortex (in vicinity of Brodman area 11)+d₄*left dorso-lateral prefrontal cortex (in vicinity of Brodman area 9))
where d₁=0.1, d₂=0.4, d₃=0.1, d₄=0.4,
whereby said values indicate each subject's engagement in features of the advertisement.

29. A method as claimed in claim 6 including the steps of applying an electrode to the scalp of each subject at a site approximately equidistant from the C₃, F₃ and F₇ sites, calculating SSVEP amplitudes and phase differences from EEG signals from said electrode at the time branding information is presented, whereby output signals indicate each subject's behavioural intent to the subject matter of the advertisement.

30. A method as claimed in claim 7 wherein the step of utilising inverse mapping determines brain activity in the left cerebral cortex in the vicinity of Brodman's areas 6, 44, 45, 46 and 47, calculating SSVEP amplitude and phase differences from said location at the time branding information is presented whereby output signals indicate each subject's behavioural intent to the subject matter of the advertisement.

31. A method as claimed in claim 1 wherein said pooled output signals are displayed graphically.

32. A method as claimed in claim 31 wherein said pooled output signals are displayed on a video monitor which simultaneously displays the advertisement being assessed.

33. A method of measuring steady-state visually evoked potential (SSVEP) of a subject including the step of applying time varying flicker signals only to the peripheral vision regions of the retina of a subject and not applying said time varying flicker signals to the centre of vision (fovea) of the subject.

34. A method as claimed in claim 33 wherein said SSVEP amplitude and phase are calculated by the equations:
$${SSVEP}_{amplitude} = \sqrt{( {A}_{n}^{2} + {B}_{n}^{2} )}$$
$${SSVEP}_{phrase} = a tan ( \frac{{B}_{n}}{{A}_{n}} )$$
where: aₙ and bₙ are cosine and sine Fourier coefficients calculated by the equations:
$${a}_{n} = \frac{1}{S Δ τ} {\sum }_{i = 0}^{S - 1} f ( nT + i Δ τ ) cos ( \frac{2 π}{T} ( nT + i Δ τ ) )$$
$${b}_{n} = \frac{1}{S Δ τ} {\sum }_{i = 0}^{S - 1} f ( nT + i Δ τ ) sin ( \frac{2 π}{T} ( nT + i Δ τ ) )$$
where:
aₙ and bₙ are the cosine and sine Fourier coefficients respectively where;
n represents the nth flicker stimulus cycle;
S is the number of samples per flicker stimulus cycle;
Δτ is the time interval between samples;
T is the period of one cycle;
f(nT+iΔτ) is the EEG signal (raw or pre-processed using ICA) obtained from said predetermined scalp sites;
where: Aₙ and Bₙ are overlapping smoothed Fourier coefficients calculated by using the equation:
$${A}_{n} = {\sum }_{i = 1}^{i = N} {a}_{n + i} / N$$
$${B}_{n} = {\sum }_{i = 1}^{i = N} {b}_{n + i} / N$$

35. A system for quantitatively assessing the effectiveness of an audiovisual, visual or audio advertisement including:
display means for presenting the advertisement to a plurality of subjects, the advertisement having a sequence of audiovisual, visual or audio features which occur as a function of time;
means for obtaining, during presentation of the advertisement, EEG signals from said at least one subject from predetermined scalp sites of said subjects; and
means for calculating SSVEP amplitudes and/or phase differences from signals obtained from the predetermined sites in order to obtain output signals which represent said predetermined psychological states of said at least one subject to said features as a function of time, to thereby enable quantitative assessment of said subjects' responses to said features of the advertisement in order to assess the effectiveness of the features of the advertisement.

36. A method of producing an audiovisual advertisement including the steps of:
producing an early version of the advertisement such as a story-board or an animatic or a finished version of the advertisement;
quantitatively assessing the effectiveness of the early or finished version of the advertisement in accordance with the method as claimed claim 1; and
editing the early or finished version of the advertisement to modify features of the advertisement which are assessed to be unsatisfactory in order to produce an improved advertisement.
